(19) United States
(12) Patent Application Publication
(10) Pub. No.: US 2004/0146537 A1
(43) Pub. Date: Jul. 29, 2004

(54) Oily wax matrix suspension formulation comprising pharmacologically active agents

(76) Inventors: Ramachandran Radhakrishnan, Bangalore (IN); Nehru Babu Gaddipati, Somerset, NJ (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/353,259

(22) Filed: Jan. 28, 2003

(51) Int. Cl. A61K 9/00

(52) U.S. Cl. 424/400

(57) ABSTRACT

Embodiments of the present invention relate to an oily wax matrix suspension pharmaceutical formulation for oral administration through a soft gelatin capsule drug delivery device, where the pharmaceutical formulation comprises non-steroidal anti-inflammatory drugs (NSAIDs) including Ketoprofen, Naproxen, and Naproxen Sodium salt form as the active ingredient. The active pharmaceutical ingredient is embedded in an oily matrix, which also comprises a surfactant, a viscosity enhancer and a suspending agent.

### BACKGROUND OF THE INVENTION

1. Field of the Invention

In general, the invention relates to suspension formulations of pharmacologically active agents. More particularly, the invention relates to an oral administrable oily wax matrix suspension formulation comprising non-steroidal anti-inflammatory drugs (NSAIDs) including Ketoprofen, Naproxen, and Naproxen Sodium, encapsulated into soft gelatin capsules.

2. Description of the Related Art

Ketoprofen and Naproxen are derivatives of propionic acid and Naproxen Sodium is sodium salt of Naproxen. Ketoprofen, Naproxen and its sodium salt are non-steroidal anti-inflammatory drugs (NSAID), with analgesic (pain relieving) and antipyretic (fever reducing) properties, which are commonly used to relieve pain and to treat inflammatory conditions. Ketoprofen and Naproxen are in the form of white powders or crystals, which are practically insoluble in water; Naproxen Sodium is soluble in water.

Patient compliance is improved if a soft gelatin capsule is used for drug administration because of its soft elastic character making it easier to swallow compared to conventional tablets or hard gelatin capsules. Furthermore, since the dosage form is generally swallowed, it is unnecessary to flavor or otherwise mask any unpleasant taste of the active pharmaceutical ingredients. Finally, unlike tablets, soft gelatin capsules do not chip or powder, thus keeping intact the entire dosage of the active ingredient(s).

Filled one-piece soft gels have been widely known and used for many years for a variety of purposes. Soft gels have properties which are different from conventional telescoping two-piece hard shell capsules, making them capable of retaining liquid fill material. Typically, soft gels are used to contain orally consumable materials such as vitamins and pharmaceutical compositions in a liquid vehicle or carrier.

U.S. Pat. No. 4,944,949 to Story, Michael J. et al. describes a micelle-forming composition of non-steroidal anti-inflammatory drugs including ketoprofen, naproxen or ibuprofen formulated with surfactants such as polyethoxylated nonionics.

U.S. Pat. No. 5,202,129 to Samejima, et al. describes a process for micronizing a slightly soluble drug, comprising grinding of the drug in the presence of a sugar or sugar alcohol of a lower molecular weight.

U.S. Pat. No. 5,624,682 to Dondi, et al. describes a stable pharmaceutical composition of ketoprofen comprising a carrier, such as polyethylene glycol.

U.S. Pat. No. 6,238,703 to Jan, et al. describes a control release analgesic dosage form including ketoprofen or naproxen comprising a binding agent and a coating with an enteric polymer, a water insoluble second polymer and a lubricant.

A composition including soybean oil, yellow beeswax and lecithin has been disclosed in the U.S. Pat. No. 6,309,677 to Horvath et al. However, the active in this disclosure are extracted carotenoides.

U.S. Pat. No. 5,175,002 addresses a suspension formulation comprising soybean oil, lecithin and wax with Amantidine Hydrochloride as the active ingredient.

U.S. Pat. No. 6,197,347 to Jan, et. al describes an oral dosage formulation in the form of a tablet or capsule containing pellets comprising a non-steroidal anti-inflammatory drug, preferably propionic acid derivatives such as ibuprofen, ketoprofen, naproxen, indoprofen with coating of a mixture of an enteric polymer, a water insoluble polymer and lubricant.

U.S. Pat. No. 5,376,688 to Morton, et al. describes a pharmaceutical formulation of acidic, basic or amphoteric pharmaceutical agents including, ketoprofen, naproxen, suitable for encapsulation in gelatin capsule comprising the acidic pharmaceutical agent, a hydroxide species and a solvent system, the solvent system consisting from the group of diethylene glycol monoethyl ether, polyglycerol oleate and mixture there of.

U.S. Pat. No. 5,431,916 to White et al. describes a pharmaceutical composition in a soft gelatin capsule comprising at least one pharmaceutically acceptable active including ketoprofen, naproxen formulated in a mixture of a tri-ester and polyvinylpyrrolidone and a process for manufacturing such pharmaceutical composition.

U.S. Pat. No. 5,141,961 to Coapman et al. describes a soft gelatin capsule comprising one difficult pharmaceutical including naproxen in a mixture of polyethylene glycol and polyvinyl pyrrolidone

### SUMMARY OF THE INVENTION

In order to provide better patient compliance, embodiments of the present invention include a pharmaceutical formulation comprising a soft gelatin capsule formulation containing pharmacologically active agents, particularly the suspension formulation of non-steroidal anti-inflammatory drugs (NSAIDs) including Ketoprofen, Naproxen and Naproxen Sodium. In preferred embodiments, suspension formulations provide stability of the drugs over prolonged period of time, and uniform distribution of the active drug. A further increase in the viscosity of the solid drug form is achieved by using a suspending agent. Suspension formulations preferably use one or more suspending agents to make a substantially homogenous dispersion of the active in the fill preparation, and thus allow dosing uniformity when the suspension is filled into capsules.

One embodiment of the present invention provides for soft gelatin capsules of a pharmaceutical formulation for oral administration comprising about 12.5-75 mg by weight of Ketoprofen, about 5-20 mg by weight of yellow beeswax, about 1-15 mg by weight of lecithin, about 5-25 mg partially hydrogenated vegetable oil, about 1-15 mg colloidal silicon dioxide and about 100-300 mg by weight of soybean oil.

Another embodiment of the present invention provides for soft gelatin capsule of a pharmaceutical formulation for oral administration comprising about 12.5-75 mg by weight of Ketoprofen, about 5-20 mg by weight of yellow beeswax, about 5-25 mg partially hydrogenated vegetable oil, about 1-15 mg by weight of lecithin and about 100-500 mg by weight of soybean oil.

Other embodiments of the present invention include: soft gelatin capsules of a pharmaceutical formulation for oral administration comprising about 250 mg or about 375 mg by weight of Naproxen or about 220 mg or about 275 mg by weight of Naproxen Sodium, about 1-15 mg by weight of yellow beeswax, about 5-35 mg by weight of lecithin and about 100-500 mg by weight of soybean oil; and soft gelatin capsules of a pharmaceutical formulations comprising about 500 mg by weight of Naproxen and about 550 mg by weight of Naproxen Sodium about 1-30 mg by weight of yellow beeswax, about 5-50 mg by weight of lecithin and about 100-500 mg by weight of soybean oil.

Other embodiments include methods of making an oral pharmaceutical formulation comprising preparing an oily matrix consisting of soybean oil and partially hydrogenated vegetable oil, the oily blend is heat treated with beeswax, to have the beeswax dissolved into the matrix, the steps further comprises blending lecithin to the oily matrix and mixing the active pharmaceutical ingredient into the matrix. Colloidal silicon dioxide is added to the complex to form a homogeneous blend and the resultant pharmaceutical complex is enclosed into a capsule of about 12.5-75 mg by weight of Ketoprofen, about 5-20 mg by weight of yellow beeswax, about 1-15 mg by weight of lecithin, about 5-25 mg partially hydrogenated vegetable oil, about 1-15 mg colloidal silicon dioxide and about 100-300 mg by weight of soybean oil.

Additional embodiments include methods of making a pharmaceutical formulation comprising preparing an oily matrix consisting of soybean oil and partially hydrogenated vegetable oil, the oily blend is heat treated with beeswax, to have the beeswax dissolved into the matrix, further comprising blending lecithin to the oily matrix and mixing the active pharmaceutical ingredient into the matrix, resulting in a formulation of about 12.5-75 mg by weight of Ketoprofen, about 5-20 mg by weight of yellow beeswax, about 5-25 mg partially hydrogenated vegetable oil, about 1-15 mg by weight of lecithin and about 100-500 mg by weight of soybean oil.

In accordance with another embodiment, the invention includes methods of making a pharmaceutical formulation comprising preparing an oily matrix consisting of soybean oil and beeswax, blending lecithin to the oily matrix, mixing an active pharmaceutical ingredient into the matrix and enclosing the oily matrix embedded pharmaceutical complex into a capsule. Also is the preferred embodiment to dispose pharmaceutical complex into a soft gelatin drug delivery device, wherein used is about 250 mg or about 375 mg by weight of Naproxen or about 220 mg or about 275 mg by weight of Naproxen Sodium about 1-15 mg by weight of yellow beeswax, about 5-35 mg by weight of lecithin and about 100-500 mg by weight of soybean oil.

Another method of making an oral pharmaceutical formulation according to the invention comprises preparing an oily matrix consisting of soybean oil and beeswax, blending lecithin to the oily matrix, mixing an active pharmaceutical ingredient into the matrix and enclosing the oily matrix embedded pharmaceutical complex into a capsule. Another embodiment includes disposing the pharmaceutical complex into a soft gelatin drug delivery device comprising a formulation of about 500 mg by weight of Naproxen and about 550 mg by weight of Naproxen Sodium about 1-30 mg by weight of yellow beeswax, about 5-50 mg by weight of lecithin and about 100-500 mg by weight of soybean oil.

### DETAILED DESCRIPTION OF CERTAIN EMBODIMENTS

The present invention relates to pharmaceutical formulations comprising Ketoprofen, Naproxen and Naproxen Sodium for oral administration, where a soft gelatin capsule delivers the pharmaceutical formulation.

In accordance with one embodiment the formulation containing Ketoprofen in an oily wax matrix suspension formulation comprising yellow beeswax, partially hydrogenated vegetable oil, colloidal silicon dioxide, soybean oil and lecithin. Soybean oil has been used in the embodiment as a suspension medium and yellow beeswax as a suspending agent. Hydrogenated vegetable oil has been used as a viscosity inducing agent and colloidal silicon dioxide is used to achieve uniform dose dispersion.

In accordance with another embodiment, the formulation contains Naproxen and/or Sodium salt of Naproxen also comprises of yellow beeswax, soybean oil and lecithin. Soybean oil is used in the embodiment as a suspension medium and yellow beeswax is used as a suspending agent.

The following examples illustrate certain preferred embodiments of pharmaceutical compositions comprising Ketoprofen, Naproxen and Naproxen Sodium as the principal pharmaceutically active ingredient.

### EXAMPLES

### Example 1

| Ingredients | Composition by weight (approx.) |
|---|---|
| Ketoprofen | 12.5-75 mg |
| Yellow Beeswax | 5-20 mg |
| Lecithin, NF | 1-15 mg |
| Partially Hydrogenated vegetable Oil | 5-25 mg |
| Colloidal silicon dioxide | 1-15 mg |
| Soybean Oil, USP | 100-300 mg |

The fill above was prepared by preparing an oily matrix consisting of soybean oil and partially hydrogenated vegetable oil. In order to have beeswax dissolved into the matrix, the oily blend is heat treated with beeswax. Then lecithin is blended into the oily matrix, and the active pharmaceutical ingredient is mixed into the matrix, forming a complex. Colloidal silicon dioxide is added to the complex to form a homogeneous blend. Finally, the active ingredient was dispersed in the blend and deaerated to remove any trapped gases.

Example 2

| Ingredients | Composition by weight (approx.) |
|---|---|
| Ketoprofen | 12.5-75 mg |
| Yellow Beeswax | 5-20 mg |
| Partially Hydrogenated Vegetable Oil | 5-25 mg |
| Lecithin, NF | 1-15 mg |
| Soybean Oil, USP | 100-500 mg |

The above fill was prepared as described in Example 1.

### Example 3

| Ingredients | Composition by weight( in mg)(approx.) | | |
|---|---|---|---|
| Naproxen | 250 | 375 | 500 |
| Yellow Beeswax | 1-15 | 1-15 | 1-30 |
| Lecithin, NF | 5-35 | 5-35 | 5-50 |
| Soybean Oil, USP | 100-500 | 100-500 | 100-500 |

### Example 4

| Ingredients | Composition by weight( in mg) (approximately) | | |
|---|---|---|---|
| Naproxen Sodium | 220 | 275 | 550 |
| Yellow Beeswax | 1-15 | 1-15 | 1-30 |
| Lecithin, NF | 5-35 | 5-35 | 5-50 |
| Soybean Oil, USP | 100-500 | 100-500 | 100-500 |

The fill for Examples 3 and 4 above was prepared by heating the soybean oil to about 60-65 C. The yellow beeswax and/or hydrogenated vegetable oil was added and mixed with the soybean oil until the wax was melted and the dispersion of these ingredients was homogenous. Lecithin was then added to the mixture. While the mixture is being stirred, Naproxen or Naproxen Sodium was added in, thereby forming a homogenous dispersion of the ingredients. Finally, the blend was deaerated to remove any entrapped air.

In general, gelatin capsule sheath formulations for soft gelatin capsules comprise raw gelatin and one or more plasticizers added to adjust the hardness of the capsule. Typical plasticizers include glycerin, sorbitol and Anidrisorb 85/70. A preferred plasticizer is Anidrisorb 85/70, an aqueous solution of D-sorbitol and sorbitans. One preferred gelatin formulation for the soft gelatin capsules used in accordance with preferred embodiments includes gelatin in the range of about 40% to about 48% and a plasticizer ranging in amount from about 16% to about 35%. Another preferred plasticizer is Sorbitol BP, a non-crystallizing sorbitol solution. When either an approximately 70% non-crystallizing sorbitol solution or Anidrisorb 85/70 are used alone, the amount of plasticizer used preferably ranges from about 16% to about 35%. Capsule formulations can also include other suitable additives such as antioxidants, amino acids and coloring agents, which impart specific characteristics, including capsule aesthetics. Antioxidants include Butylated Hydroxy Anisole (BHA), Butylated Hydroxy Toluene (BHT), and citric acid, though other antioxidants such as tocopherol, tocopherylacetate, d-α-tocopheryl polyethylene glycol 1000 succinate, cysteine, ascorbic acid, calcium propionate, sorbic acid, potassium sorbate, ethoxyquin, lactic acid, benzoic acid, sodium benzoate, ethyl-p-hydroxybenzoate, and propyl-p-hydroxybenzoate may be used. FD&C dyes and D & C dyes are examples of pharmaceutically acceptable coloring agents that may be used in preferred embodiments.

The formulation is a suspension containing Ketoprofen, Naproxen or Naproxen Sodium in a wax matrix. The solubility is not a limiting factor and the same concept can be extended to higher strengths. In the examples, soft gel capsules contain different strengths of actives, such as about 12.5-75 mg by weight of Ketoprofen, about 250 mg, about 375 mg or about 500 mg by weight of Naproxen or about 220 mg, about 275 mg or about 550 mg by weight of Naproxen Sodium are envisioned.

The following examples illustrate certain preferred embodiments of several soft-gelatin-shell Ketoprofen, Naproxen and/or Naproxen Sodium, and are not intended to limit the scope of the invention.

TABLE 1

| | 2 | 3 | 4 | 5 |
|---|---|---|---|---|
| Ingredients | Weight percent range (min-max) (mg) | | | |
| Gelatin | 38.0-46.0 | 38.0-46.0 | 38.0-46.0 | 38.0-46.0 |
| Sorbitol Solution | 14.0-25.0 | 14.0-25.0 | 14.0-25.0 | 14.0-25.0 |
| Glycine | 0.2-0.6 | 0.2-0.6 | 0.2-0.6 | 0.2-0.6 |
| BHA | 0.02-0.03 | 0.02-0.03 | 0.02-0.03 | 0.02-0.03 |
| BHT | | 0.02-0.03 | | 0.02-0.03 |
| Citric Acid | | | 0.42-0.46 | 0.42-0.46 |
| Purified water | 40.5-45.5 | 40.5-45.5 | 40.5-45.5 | 40.5-45.5 |

Certain modifications and improvements of the disclosed invention will occur to those skilled in the art without departing from the scope of, invention, which is limited only by the appended claims.

Gelatin paste preparation is carried out in a melter. The gelatin paste preparation is done by heating the gelatin with plasticizer and purified water with continuous stirring. During gelatin paste preparation, vacuum is applied to remove extra amounts of water added and to get a gelatin ribbon free from air bubbles. Colorants may be optionally added and mixed further in a stainless steel tank at 60±5° C. for 1 to 2 hours to get a uniform color distribution. The blend of the product fill and gelatin paste as obtained above are taken for encapsulation. Manufacturing of soft gelatin capsules is carried out using rotary die process. The shape of capsule may be oval, round or oblong, most preferably oval shaped with a 16 mm length. Encapsulation process is carried out at temperature below 30° C. and relative humidity below 25%.

All patents and publications mentioned in the specification arc indicative of levels of those skilled in the art to which the invention pertains. All patents and publications are herein incorporated by reference in their entireties to the same extent as if each individual publication was specifically and individually indicated to be incorporated by reference.

It will be readily apparent to one skilled in the art that varying substitutions and modifications may be made to the invention disclosed herein without departing from the scope and spirit of the invention. Thus, it should be understood that although the present invention has been specifically disclosed by preferred embodiments and optional features, modification and variation of the concepts herein disclosed may be resorted to by those skilled in the art, and that such modifications and variations are considered to be falling within the scope of the invention, which is limited only by the following claims.

What is claimed:

1. A pharmaceutical formulation comprising:
an active pharmaceutical ingredient embedded into an oily wax matrix;
the oily wax matrix comprising:
a surfactant;
a suspending agent;
a viscosity enhancer;
a dispersion medium; and
a suspension medium,
wherein said formulation is for oral administration, and wherein the active pharmaceutical ingredient is a non-steroidal anti-inflammatory drug (NSAID).

2. The pharmaceutical formulation of claim 1, wherein the formulation is a suspension formulation.

3. The pharmaceutical formulation of claim 1, wherein the surfactant is lecithin.

4. The pharmaceutical formulation of claim 1, wherein the suspending agent is yellow beeswax.

5. The pharmaceutical formulation of claim 1, wherein the viscosity-imparting agent is partially hydrogenated vegetable oil.

6. The pharmaceutical formulation of claim 1, wherein the dispersion medium is colloidal silicon dioxide.

7. The pharmaceutical formulation of claim 1, wherein the suspension medium is selected from the group consisting of almond oil, babassu oil, borage oil, blackcurrant seed oil, canola oil, castor oil, coconut oil, corn oil, cottonseed oil, evening primrose oil, grape seed oil, groundnut oil, mustard seed oil, olive oil, palm oil, palm kernel oil, peanut oil, rapeseed oil, safflower oil, sesame oil, shark liver oil, sunflower oil, hydrogenated castor oil, hydrogenated coconut oil, hydrogenated palm oil, hydrogenated soybean oil, hydrogenated vegetable oil, hydrogenated cottonseed and castor oil, partially hydrogenated soybean oil, soy oil, glyceryl tricaproate, glyceryl tricaprylate, glyceryl tricaprate, glyceryl triundecanoate, glyceryl trilaurate, glyceryl trioleate, glyceryl trilinoleate, glyceryl trilinolenate, glyceryl tricaprylate/caprate, glyceryl tricaprylate/caprate/laurate, glyceryl tricaprylate/caprate/linoleate, glyceryl tricaprylate/caprate/stearate, saturated polyglycolized glycerides, linoleic glycerides, caprylic/capric glycerides, modified triglycerides, fractionated triglycerides, and mixtures thereof.

8. The pharmaceutical formulation of claim 7, wherein the suspension medium is soybean oil.

9. The pharmaceutical formulation of claim 1, wherein the active pharmaceutical ingredients are non-steroidal anti-inflammatory drugs (NSAIDs).

10. The pharmaceutical formulation of claim 9, wherein the active pharmaceutical ingredients are Ketoprofen, Naproxen or Naproxen Sodium.

11. A pharmaceutical formulation comprising:
about 12.5-75 mg by weight of Ketoprofen;
about 5-20 mg by weight of yellow beeswax;
about 1-15 mg by weight of lecithin, NF;
about 5-25 mg by weight of Partially Hydrogenated Vegetable Oil; and
about 100-500 mg by weight of Soybean Oil, USP,
wherein said formulation is for oral administration.

12. The pharmaceutical formulation of claim 11, further comprising about 1-15 mg by weight of Colloidal silicon dioxide and wherein the amount of Soybean Oil, USP is about 100-300 mg by weight.

13. A method for preparing a pharmaceutical formulation comprising:
preparing an oily matrix consisting of soybean oil, beeswax and partially hydrogenated vegetable oil;
blending lecithin and silicon dioxide to said oily matrix;
mixing an active pharmaceutical ingredient into the said matrix; and
encapsulating the oily matrix-embedded pharmaceutical complex into a capsule for oral administration.

14. The method for preparing the pharmaceutical formulation of claim 13, wherein the said active pharmaceutical ingredient is Ketoprofen.

15. The method for preparing the pharmaceutical formulation of claim 13, wherein the said capsule is a soft gelatin capsule.

16. A pharmaceutical formulation for oral administration comprising:
an active ingredient of Naproxen, in an amount by weight selected from the group consisting of about 250 mg, about 375 mg, and about 500 mg by weight, or of Naproxen Sodium, in an amount by weight selected from the group consisting of about 220 mg, about 275 mg, and about 550 mg;
yellow beeswax, in about 1-0.30 mg of weight;
lecithin, in about 5-50 mg by weight; and
soybean oil, in about 100-500 mg by weight.

17. The pharmaceutical formulation of claim 16, wherein
the active ingredient is selected from the group consisting of Naproxen, in an amount by weight of 250 mg, Naproxen in an amount by weight of about 375 mg, Naproxen Sodium in an amount of by weight of about 220 mg, and Naproxen Sodium in an amount by weight of about 275 mg;
the yellow beeswax is in an amount by weight of about 1-15 mg; and
the amount of lecithin is in an amount by weight of about 5-35.

18. The pharmaceutical formulation of claim 17, wherein the active ingredient is Naproxen.

19. The pharmaceutical formulation of claim 17, wherein the active ingredient is Naproxen Sodium.

20. The pharmaceutical formulation of claim 16, wherein the active ingredient is Naproxen in an amount by weight of about 500 mg or Naproxen Sodium in an amount by weight of about 550 mg.

21. The phamaceutical formulation of claim 20, wherein the active ingredient is Naproxen.

22. The pharmaceutical formulation of claim 20, wherein the active ingredient is Naproxen Sodium.

23. A method for preparing a pharmaceutical formulation consisting of:
preparing an oily matrix consisting of soybean oil and beeswax;
blending lecithin to said oily matrix;
mixing an active pharmaceutical ingredient into the said matrix; and
encapsulating the oily matrix-embedded pharmaceutical complex into a capsule.

24. The method of claim 23, wherein the said active pharmaceutical ingredient is Naproxen or Naproxen Sodium.

25. The method of claim 23, wherein the said capsule is a soft gelatin capsule.
